(19) United States
(12) Patent Application Publication
(10) Pub. No.: US 2025/0000907 A1
(43) Pub. Date: Jan. 2, 2025

(54) ENGINEERED IMMUNE CELLS WITH REDUCED SIRT6 EXPRESSION

(71) Applicant: H. LEE MOFFITT CANCER CENTER AND RESEARCH INSTITUTE, INC., Tampa, FL (US)

(72) Inventors: Sungjune KIM, Tampa, FL (US); Imene HAMAIDI, Tampa, FL (US)

(21) Appl. No.: 18/712,431

(22) PCT Filed: Nov. 22, 2022
(86) PCT No.: PCT/US2022/050701

§ 371 Date: May 22, 2024

Related U.S. Application Data
(60) Provisional application No. 63/281,931, filed on Nov. 22, 2021.

(51) Int. Cl. A61K 35/17; A61K 39/00; C07K 14/725; C12N 5/078

(52) U.S. Cl. CPC A61K 35/17; A61K 39/4611; A61K 39/4631; C07K 14/7051; C12N 5/0634; C12N 2510/00

(57) ABSTRACT

Disclosed are methods of making engineered cells comprising reduced Sirt6 expression as well as the cells that are the products of said methods. Thus, in one aspect, disclosed herein are engineered lymphocytes comprising reduced Sirt6 expression. Also disclosed herein are methods of treating cancer in a subject that involves collecting lymphocytes, such as tumor infiltrating lymphocytes (TILs), from the subject, treating the lymphocytes ex vivo to inhibit Sirt6 expression, and transferring the modified lymphocytes to a subject with a cancer.

This invention was made with government support under Grant No. R37CA248298 awarded by the National Institutes of Health. The government has certain rights in the invention.

### I. BACKGROUND

Cancer immunotherapies are leading the paradigm shift in cancer care. Despite such breakthroughs, many cancers fail to respond to these therapies or maintain a durable response. This has been linked to the exhaustion of tumor-infiltrating lymphocytes (TILs), characterized by a loss of T cell effector functions. There is a growing consensus indicating that dysregulated metabolism is a key driver of TIL exhaustion within the tumor microenvironment (TME). Accordingly, metabolic manipulation of T cells to optimally combat tumor cells is an attractive strategy to restore anti-tumor immunity. What is needed are immunotherapies that do not suffer the problems of existing technology.

### II. SUMMARY

Disclosed are methods and compositions related to immune cells with reduced, inhibited, decreased, and/or ablated Sirt6 expression.

In one aspect, disclosed herein are methods making a modified immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and yo T cells), natural killer (NK) cell, NK T cell, macrophage, B cell, CAR T cell, CAR NK Cell, CAR NK T cell, CAR macrophage, and/or CAR B cells) comprising obtaining lymphocytes (including, but not limited to lymphocytes obtained from a subject with cancer); and treating the lymphocytes with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

Also disclosed herein are methods of making a modified immune cell of any preceding aspect, wherein the lymphocytes are genetically modified by inserting a chimeric receptor (such as, for example, a chimeric antigen receptor (CAR)) into the genome of the cell at a location that disrupts expression or activity of an endogenous Sirt6 protein.

In one aspect, disclosed herein are methods of making a modified immune cell of any preceding aspect, wherein the modified immune cell is further modified by treating the lymphocytes with a Sirt2 inhibitor or genetically modifying the lymphocytes to inhibit or ablate Sirt2 expression (such as, for example, a small molecule such as AGK2, AK-1, SirReal2, Tenovin-6, Thiomyristoyl, AEM1, or AEM2; siRNA; shRNA; antisense oligonucleotide; or gRNA). In some aspects, the lymphocytes are genetically modified by inserting a chimeric receptor (for example, a CAR) into the genome of the cell at a location that disrupts expression or activity of an endogenous Sirt2 protein.

Also disclosed herein are therapeutic cells produced by the method of any preceding aspect. For example disclosed herein are engineered immune cells (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and yo T cells), natural killer (NK) cell, NK T cell, macrophage, B cell, CAR T cell, CAR NK Cell, CAR NK T cell, CAR macrophage, and/or CAR B cells) modified to have reduced, inhibited, and/or ablated Sirt6 expression (including cells modified by a small molecule such as OSS_128167 (OSS), siRNA, shRNA, antisense oligonucleotide, or gRNA). In one aspect, said engineered immune cells can further comprise reduced, inhibited, and/or ablated Sirt2 expression.

In one aspect disclosed herein are methods of treating, inhibiting, reducing, decreasing, amelioration, and/or preventing a cancer and/or metastasis (such as, for example, hepatocellular carcinoma and hematologic malignancies) in a subject, comprising administering to the subject a therapeutically effective amount of the therapeutic cells (such as, for example, any TIL, MIL, CAR T cell, CAR NK Cell, CAR NK T cell, CAR macrophage, and/or CAR B cell disclosed herein with reduced, inhibited, decreased, and/or ablated Sirt6 expression including, but not limited to any of the aforementioned cells further comprising reduced, inhibited, decreased, and/or ablated Sirt2 expression) disclosed herein. In some aspect, the methods can further comprise the administration of an anticancer agent (such as, for example, a checkpoint inhibitor including, but not limited to a checkpoint inhibitor comprising an anti-PD-1 antibody, anti-PD-L1 antibody, anti-CTLA-4 antibody, or a combination thereof).

Also disclosed herein are methods of increasing proliferation, survival, and/or effector expression of an immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

In one aspect, disclosed herein are methods of increasing effector molecule expression (such as, for example IFNγ and/or granzyme B) in an immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

Also disclosed herein are methods of increasing glycolysis, mitochondrial respiration, glutaminolysis, fatty acid oxidation, lipogenesis metabolism of an immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the immune cells to inhibit or ablate Sirt6 expression (such as, for example, a small molecule such as OSS_128167 (OSS), siRNA, shRNA, antisense oligonucleotide, or gRNA).

In one aspect, disclosed herein are methods of increasing the transcription of glycolytic targets, glutaminolysis pathways targets and fatty acid oxidation targets in an immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

Also disclosed herein are methods of increasing the transcriptional activity of c-Myc and HIF-1a transcription factors in an immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1: Hypothesis and study design. Anticipated mechanisms of action of Sirt6 and strategies to demonstrate Sirt6 as an immunotherapeutic target are depicted.

FIG. 2: Metabolic switch during T cell activation and maturation. TN: naïve T cells, TEFF: effector T cells, TM: memory T cells, TIL: Tumor-infiltrating lymphocytes, TME: Tumor micro-environment.

FIG. 3: Sirtuins in the immune system. A, WB of Sirt1-3,5-7 expression in activated CD3+ T cells from WT vs. Sirt2−/− mice. ECAR measured by Seahorse analyzer (B) and IFN-γ ELISPOT assay (C) in stimulated CD8+ T cells from WT vs. Sirt2−/− Pmel mice treated with vehicle (Ctl), or Sirt6 inhibitor (Sirt6i). *P<0.05, **P<0.01, ****P<0.0001.

FIG. 4: Model of Sirt6 function. Sirt6 co-represses HIF-1α activity by deacetylating histone H3K9.

FIG. 5: Sirt6 expression is induced upon activation, maturation and within the TME. A, Sirt6 expression in Ova-activated CD4+OT-II and gp100-activated CD8+ Pmel T cells by WB. B, Sirt6 expression in CD8+CD44high vs. CD8+CD44low subsets from spleen and lymph node. C, Sirt6 expression levels in TN, TEFF, and TM subsets of CD4+OT-II T cells and CD8+ Pmel T cells by WB. D, Sirt6 expression in CD4+CD44high and CD8+CD44high TIL vs. splenic T cells from B16F10-challenged mice.

FIG. 6: Sirt6 inhibition increases metabolic activity of T cells. ECAR (A) and OCR (G) measured by Seahorse analyzer In CD3-stimulated CD3+ T cells treated with vehicle (DMSO), 25 μM (C1) or 50 μM (C2) of Sirt6 inhibitor (OSS). Efficiency of Sirt6 knockdown (B) and Sirt6 knockout (C) are shown by WB. ECAR (D) and OCR (H) of CD3-stimulated CD3+ T cells transduced with shCt1, shSirt6 #1 or #2. ECAR (E) and OCR (I) of CD3-stimulated Sirt6fl/fl T cells transduced with recombinase Cre lentivectors. F, ECAR measured by Seahorse analyzer of CD3+ TILs isolated from B16F10 tumor nodules and treated with DMSO or OSS. ****P<0.0001. Gluc: glucose, Oligo: Oligomycin, 2-DG: 2-deoxyglucose, R/A: Rotenone/Antimycin A. ECAR: mpH/min/μg protein, OCR: pmol/min/μg protein.

FIG. 7: Sirt6 targeting improves T cell effector functions ex vivo. A, CFSE dilution in OT-II and Pmel T cells. Intracellular granzyme B expression (B), IFN-γ ELISPOT assay (C), and LDH cytotoxic assay (D) of Pmel T cells stimulated with gp100 and treated with vehicle (DMSO) or Sirt6 inhibitor (OSS). Intracellular perforin expression (E) and LDH cytotoxic assay (F) of CD8+ Pmel T cells transduced with shCt1, shSirt6 #1 or #2. G, IFN-γ ELISPOT assay of B16f10 TILs after re-challenge with B16F10 cells with DMSO or OSS. *P<0.05, **P<0.01, ***P<0.001, ****P<0.0001.

FIG. 8: Sirt6 directly interacts with c-Myc and HIF-1α, and regulates the transcription of the glycolytic and glutaminic pathways' targets. A, c-Myc and HIF-1α expression levels in CD3+ (upper), CD4+OT-II and CD8+ Pmel (lower) T cells at Day0 and following TCR ligation (Day 1-3). B, Sirt6 binding with H3K9, c-Myc, HIF-1α in activated CD3+ T cell by IP-IB. C, Reverse IP-IB. D, c-Myc, HIF-1α and acH3K9 expression levels following Sirt6 inhibition. Acetylation levels of H3K9 following Sirt6 gene silencing (E) and gene knockout (F). mRNA levels of Glut1, HK, PFKP and LDHA in activated CD3+ T cells following Sirt6 pharmacologic inhibition (G), gene knockdown (H) or gene knockout (I). J, Protein levels of Glut1, HK, PFKP and LDHA in activated CD3+ T cells following Sirt6 inhibition. mRNA levels of ASCT2 and GLS in activated CD3+ T cells following Sirt6 pharmacologic inhibition (K), gene knockdown (L) or gene knockout (M). *P<0.05, **P<0.01, ***P<0.001.

FIG. 9: Sirt6 blockade enhances metabolic fitness and effector functions of human NSCLC TILs. A-F, Primary human T cells were electroporated with vehicle (Ctl), Cas9-Atto550-crRNA non targeting control (NTC) or Cas9-Atto550-crRNA targeting Sirt6 (crSirt6). A, Detection of Atto550+ T cells by flow cytometry. B, Detection of Sirt6 mutant by PCR. 1kpb represents Sirt6 wild type. C, Detection of Sirt6 deletion by Wb. ECAR (D) and OCR (F) measured by Seahorse analyzer. E, Protein levels of Glut1, HK, PFKP and LDHA by Wb. G-J, Human TILs isolated from tumor biopsies of NSCLC patients were treated with Sirt6 inhibitor (OSS) or Vehicle (DMSO), H, ECAR measured by Seahorse Analyzer of 1 patient. Box plot of basal glycolysis, glycolytic capacity (I) and IFN-γ (J) from 6 NSCLC patient TILs. K, Cytotoxic activities by LDH assay of human NSCLC TILs treated vehicle or OSS and then co-cultured with their autologous tumor cells. *P<0.05, **P<0.01.

FIGS. 10A, 10B, and 10C show that Sirt6 deficiency enhances anti-tumor immunity in mice. FIG. 10A shows the number of lung metastatic nodules in Sirt6fl/fl vs. Sirt6fl/f1XCre mice i.v. challenged with B16F10 cells. FIG. 10B shows Subcutaneous B16F10 tumor growth curves of Sirt6fl/fl vs. Sirt6fl/f1XCre mice. FIG. 10C shows the number of lung metastatic nodules in NSG mice i.v. challenged with B16F10 cells and adoptively transferred with WT vs. Sirt64-CD8+ Pmel T cells.

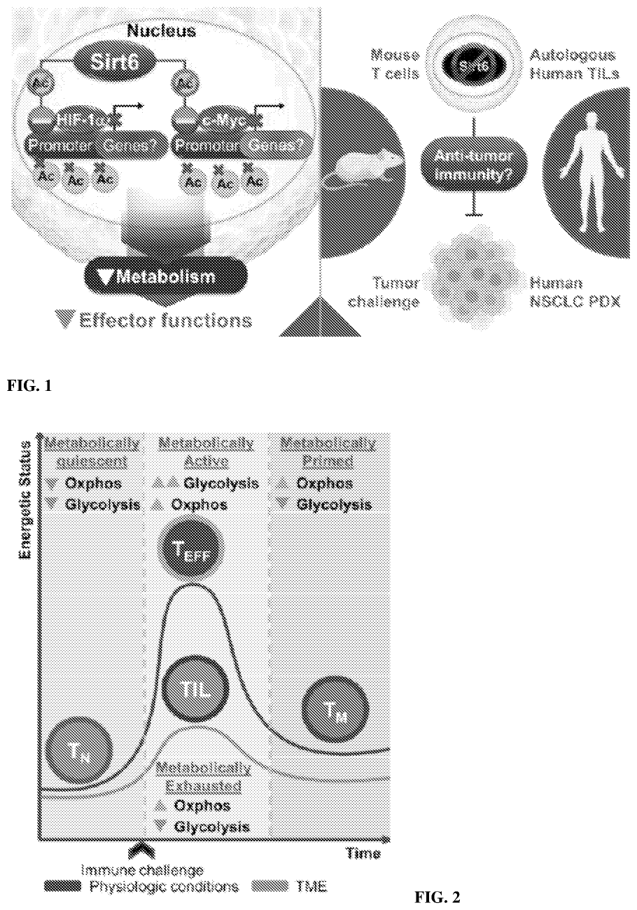

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular cell with reduced Sirt6 expression is disclosed and discussed and a number of modifications that can be made to a number of molecules including the cell with reduced Sirt6 expression are discussed, specifically contemplated is each and every combination and permutation of cell with reduced Sirt6 expression and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Cancer immunotherapies are leading the paradigm shift in cancer care. Despite such breakthroughs, many cancers fail to respond to these therapies or maintain a durable response. This has been linked to the exhaustion of tumor-infiltrating lymphocytes (TILs), characterized by a loss of T cell effector functions. There is a growing consensus indicating that dysregulated metabolism is a key driver of TIL exhaustion within the tumor microenvironment (TME). Accordingly, metabolic manipulation of T cells to optimally combat tumor cells is an attractive strategy to restore anti-tumor immunity. Recently, we have identified Sirtuin 2 (Sirt2), an NAD+ dependent histone deacetylase, as a negative regulator of T cell metabolism and we found that Sirt2 blockade in TILs leads to superior metabolic and anti-tumor activity. Mechanistically, Sirt2, a cytosolic protein, is induced following antigen stimulation and negatively impacts the acetylation and the activity of key metabolic enzymes. Interestingly, among other sirtuin family members, we observed an induction of Sirt6 expression as well following T cell activation. Sirt6 is a nuclear protein with an exclusive histone deacetylase activity that represses gene transcription via chromatin closing.

The studies herein reveal that Sirt6 is an epigenetic suppressor of T cell metabolic activity and effector functions. The findings supporting this concept are as follows: First, Sirt6 directly interacts with Histone H3K9, c-Myc and HIF-1α; and Sirt6 inhibition increases the acetylation of H3K9 and the transcription of key glycolytic targets in activated T cells. Second, Sirt6 expression is induced within the metabolically-restricted TME in mouse melanoma TILs; and Sirt6 inhibition increases TILs' glycolytic flux. Third, Sirt6 inhibition endows T cells with superior effector functions. Finally, inhibition of Sirt6 in human TILs from non-small cell lung cancer (NSCLC) patients improves their metabolic activity and effector functions ex vivo. Collectively, these data suggest that Sirt6 is a novel metabolic immune checkpoint providing a mechanistic rationale for new cancer treatment targeting Sirt6. Based on this scientific premise, my central hypothesis is that Sirt6 chromatin-silencer activity restrains the metabolic activity and effector response of T cells at tumor bed, thus facilitating cancer immune escape.

Naïve T (TN) cells are metabolically quiescent, maintain a low rate of glycolysis and rely on oxidative phosphorylation (OxPhos) to produce ATP. Following antigenic stimulation, there is a switch toward a larger reliance on aerobic glycolysis that is required for rapid proliferation and specialized effector functions of effector T (TEFF) cells. A shift from OxPhos to aerobic glycolysis represents the hallmark of activated T cells. Glutamine uptake and glutaminolysis are also upregulated in activated T cells. Glutamine fuels OxPhos and is a conditionally essential amino acid in proliferating cells. The transcription factors c-Myc and HIF-1α are crucial for T cell metabolic reprogramming. They coordinately activate the transcription of genes required for aerobic glycolysis and glutaminolysis in activated T cells. Upon pathogen clearance, a small population of memory T (TM) cells persists. TM cells predominantly rely on OxPhos that is fueled by fatty acid oxidation (FAO). Despite their low metabolic activity, TM cells display a high spare respiratory capacity (SRC) facilitating their rapid reactivation upon a secondary immune challenge. Within the metabolically challenging TME, excess glucose consumption by tumor cells restricts T cell metabolism by preventing their metabolic switch toward aerobic glycolysis required for effective anti-tumor immunity. T cells adapt to the glucose-deprived TME by promoting FA catabolismo fuel OxPhos. This catabolic pathway partially restores T cell functions within the hypoxic and hypoglycemic TME (FIG. 2).

Histone deacetylase Class III proteins, also termed sirtuins, are NAD+-dependent deacetylases. In mammals, there are seven sirtuin members (Sirt1-7) with distinct subcellular localization and functions. Sirt1, Sirt6 and Sirt7 are localized in the nucleus, where they deacetylate histones. Sirt2 is predominantly cytosolic. Sirt3-5 are localized in mitochondria, where they modulate the oxidative stress.

Sirtuins serve as energy sensors and the idea that they might be involved in immunity was posited over a decade ago, given the link between metabolic reprogramming and T cells' effector function and fate. Indeed, each sirtuin has been demonstrated to promote the resolution of inflammation. However, the investigations on their role in adaptive immunity are very limited and utmost attention has been given to Sirt1 and Sirt3. Notably, downregulation of Sirt1 expression in CD8+ T cells led to their enhanced glycolytic and cytotoxic capacity, while Sirt3 and Sirt5 deficiency in mouse models had no impact on immune responses against bacterial and fungal infections suggesting that they may play a limited role in TEFF functions. Much less is known about the role of the other sirtuins in T cells and their impact on the immune response.

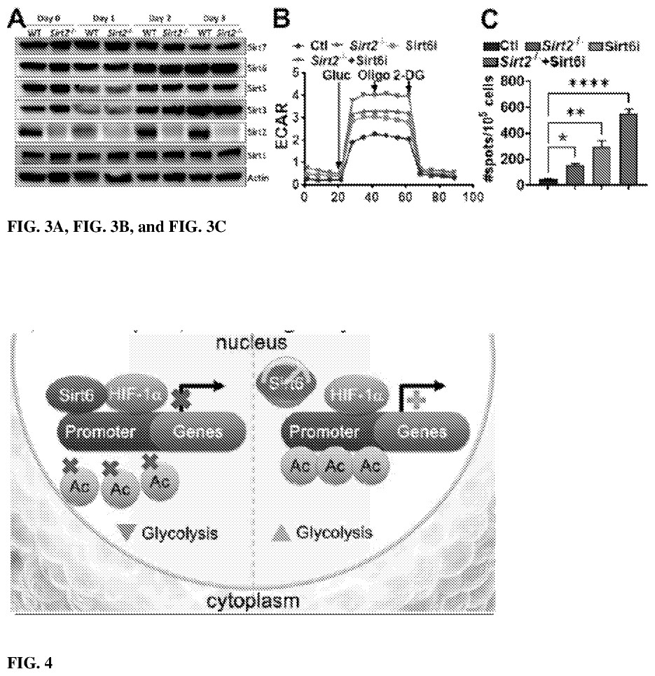

We recently uncovered a new function of Sirt2 as a master regulator of T cell metabolism. Mechanistically, Sirt2 directly interacts with metabolic enzymes to alter their acetylation status and enzymatic activities, ultimately impacting the metabolic activity and effector functions of tumor-reactive T cells. To extend our study on the role of other sirtuins in T cell-specific immune responses, our preliminary investigations revealed the induction of expression of Sirt3 and Sirt6 along with Sirt2, following TCR ligation (FIG. 3A). Since Sirt3 was broadly studied and its deficiency had limited impact on T cell effector functions, we therefore, pursued our investigation on Sirt6. Interestingly, our screening using a Sirt6 inhibitor on activated T cells showed that Sirt6 inhibition increases the metabolic activity and effector function of T cells, and these effects are potentiated with Sirt2 deficiency (FIGS. 3B&C), indicating a possible role of Sirt6 in T cell biology, independently from Sirt2 activity.

In one aspect, disclosed herein are methods making a modified immune cell ((such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and yo T cells), natural killer (NK) cell, NK T cell, macrophage, B cell, CAR T cell, CAR NK Cell, CAR NK T cell, CAR macrophage, and/or CAR B cells) comprising obtaining lymphocytes (including, but not limited to lymphocytes obtained from a subject with cancer including, but not limited to hepatocellular carcinoma and hematologic malignancies); and treating the lymphocytes with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

Also disclosed herein are methods of making a modified immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells), wherein the lymphocytes are genetically modified by inserting a chimeric receptor (such as, for example, a chimeric antigen receptor (CAR)) into the genome of the cell at a location that disrupts expression or activity of an endogenous Sirt6 protein.

In one aspect, disclosed herein are methods of making a modified immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells), wherein the modified immune cell is further modified by treating the lymphocytes with a Sirt2 inhibitor or genetically modifying the lymphocytes to inhibit or ablate Sirt2 expression (such as, for example, a small molecule such as AGK2, AK-1, SirReal2, Tenovin-6, Thiomyristoyl, AEM1, or AEM2; siRNA; shRNA; antisense oligonucleotide; or gRNA). In some aspects, the lymphocytes are genetically modified by inserting a chimeric receptor (for example, a CAR) into the genome of the cell at a location that disrupts expression or activity of an endogenous Sirt2 protein.

Sirt6 is mainly localized in the nucleus and has multiple physiological roles including cell metabolism, tumor suppression, DNA repair, and longevity. Sirt6 is a master regulator of glucose homeostasis by co-repressing HIF-1α via histone deacetylation of H3K9 at the promoters of several glycolytic genes (FIG. 4). Consequently, Sirt6 deficient cells exhibit increased aerobic glycolysis and decreased OxPhos even under normoxic conditions. In addition, Sirt6 deletion promotes FA uptake and lipogenesis. However, the precise mechanism by which Sirt6 regulates FA metabolism is not yet established. Sirt6 has been shown to be both, a tumor promoter and a tumor suppressor depending on the histologic contexts, indicating the requirement for a careful patient selection when clinically targeting Sirt6 in cancer.

Currently, the studies on Sirt6 in the immune system are insufficient. Sirt6 was reported as suppressor of inflammation via repressing NF-κB and c-JUN pathways within the myeloid compartment, and Sirt6 knockout (Sirt6^{-/-}) in mice caused chronic inflammation in several organs. However, the precise role of Sirt6 in T cell biology remains elusive.

Notwithstanding the independence of Sirt6 and Sirt2, it is understood that a concurrent reduction in Sirt6 and Sirt2 can have a synergistic effect on cancer cells. Accordingly, also disclosed herein are therapeutic cells produced by the methods disclosed herein. For example disclosed herein are engineered immune cells (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) modified to have reduced, inhibited, and/or ablated Sirt6 expression (including cells modified by a small molecule such as OSS_128167 (OSS), siRNA, shRNA, antisense oligonucleotide, or gRNA). In one aspect, said engineered immune cells can further comprise reduced, inhibited, and/or ablated Sirt2 expression.

### 1. Chimeric Antigen Receptors (CAR)

In some cases, the lymphocytes also expresses a chimeric receptor. In some embodiments, the chimeric receptor comprises a chimeric antigen receptor (CAR) polypeptide.

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28,41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. NatRev Cancer. 2016 Aug. 23; 16 (9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, Ten CAR, Dual CAR, or SCAR.

TRUCKs (T cells redirected for universal cytokine killing) co-express achimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (Tan CAR) T cell expresses a single CAR consisting of two linked single-chain variable fragments (scFvs) that have different affinities fused to intracellular co-stimulatory domain(s) and a CD3s domain. Tan CAR T cell activation is achieved only when target cells co-express both targets.

A dual CAR T cell expresses two separate CARs with different ligand binding targets; one CAR includes only the CD3s domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3s, CD30, CD3y, CD3£, CD32 (Fc gamma RIIa), DAP10, DAP12, CD79a, CD79b, FcyRIy, FcyRIIIy, Fc£RIγ (FCERIB), and Fc£RIγ (FCERIG).

In particular embodiments, the intracellular signaling domain is derived from CD3 zeta (CD3s) (TCR zeta, GenBank acc no. BAG36664.1). T-cell surface glycoprotein CD3 zeta (CD3s) chain, also known as T-cell receptor T3 zeta chain or CD247 (Cluster of Differentiation 247), is a protein that in humans is encoded by the CD247 gene.

First-generation CARs typically had the intracellular domain from the CD3s chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells.

More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3s signaling domain by itself or combined with any other desired cytoplasmic domain(s) useful in the context of the CAR of the invention. For example, the cytoplasmic domain of the CAR can comprise a CD3s chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4 (2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, 1λ2R gamma, 1λ7R a, ITGA1, V2A1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (VH) and light (VL) chain. Each VH and VL sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-ab1, HER2, α-human chorionic gonadotropin, alphafetoprotein (AFP), ALK, CD19, TIM3, cyclin B1, lectin-reactive AFP, Fos-related antigen 1, ADRB3, thyroglobulin, EphA2, RAGE-1, RU1, RU2, SSX2, AKAP-4, LCK, OY-TES1, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIB1, BORIS, prostase, prostate-specific antigen (PSA), PAX3, PAP, NY-ESO-1, LAGE-1a, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folatereceptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUC1, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, p180erbB-3, c-met, nm-23H1, PSA, CA 19-9, CA 72-4, CAM17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### 2. Immune Effector Cells

As noted above, disclosed herein are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs).

Once activated, they divide rapidly and secrete small proteins called cytokines that regulate or assist in the active immune response. These cells can differentiate into one of several subtypes, including TH1, TH2, TH3, TH17, TH9, or THH, which secrete different cytokines to facilitate a different type of immune response.

Cytotoxic T cells (Tc cells, or CTLs) destroy virally infected cells and tumor cells, and are also implicated in transplant rejection. These cells are also known as CD8+ T cells since they express the CD8 glycoprotein at their surface.

These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (T_{reg} cells), formerly known as suppressor T cells, are crucial for the maintenance of immunological tolerance. Their major role is to shutdown T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8+T lymphocytes. In some embodiments, the T cells comprise yo T cells, which possess a distinct T-cell receptor (TCR) having one y chain and one o chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### 3. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, PA 1995.

### 4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) CRISPR/Cas9

In general, “CRISPR system” or “CRISPR integration system” refers collectively to transcripts and other elements involved in the expression of or directing the activity of CRISPR-associated “Cas” genes. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

Endonuclease/RNPs (for example, a Cas9/RNP) are comprised of three components, recombinant endonuclease protein (for example, a Cas9 endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of a RNA that can hybridize to a target sequence complexed complementary repeat RNA (crRNA) and trans complementary repeat RNA (tracrRNA). Accordingly, the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. These Cas9/RNPs are capable of cleaving genomic targets with higher efficiency as compared to foreign DNA-dependent approaches due to their delivery as functional complexes. Additionally, rapid clearance of Cas9/RNPs from the cells can reduce the off-target effects such as induction of apoptosis.

### b) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### c) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 5. Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immuno bead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs-AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18 (5)); DIDS; Dihydorhodamine 123 (DHR); Dil (DilC18 (3)); I Dinitrophenol; DiO (DiOC18 (3)); DiR; DIR (DilC18 (7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices-agarose and polyacrylamide-provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, 1251). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis-I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., 32P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, NJ) and specialised chip designs, such as engineered microchannels in a plate (e.g., The Living Chip™, Biotrove, Woburn, MA) and tiny 3D posts on a silicon surface (Zyomyx, Hayward CA). Particles in suspension can also be used as the basis of arrays, providing they are coded for identification; systems include colour coding for microbeads (Luminex, Austin, TX; Bio-Rad Laboratories) and semiconductor nanocrystals (e.g., QDots™, Quantum Dot, Hayward, CA), and barcoding for beads (UltraPlex™, SmartBead Technologies Ltd, Babraham, Cambridge, UK) and multimetal microrods (e.g., Nanobarcodes™ particles, Nanoplex Technologies, Mountain View, CA). Beads can also be assembled into planar arrays on semiconductor chips (LEAPS technology, BioArray Solutions, Warren, NJ).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, WA) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, MA), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), rolling circle DNA amplification (Molecular Staging, New Haven CT), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, CA; Clontech, Mountain View, CA; BioRad; Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, CA; Biosite, San Diego, CA). In addition to the conventional antibodies, Fab and scFv fragments, single V-domains from camelids (VHH) or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, CO). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, CA).

Another methodology which can be used diagnostically and in expression profiling is the ProteinChip® array (Ciphergen, Fremont, CA), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, CT).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### 6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Method of Treating Cancer

Recent treatments based on immune checkpoint inhibitors can improve tumor-reactive T cell functions, however cure rates remain rare. There is a growing consensus indicating that metabolic fitness of T cells determines their function and fate; and the metabolic restriction of T cells by nutrient competition with tumor cells contributes to cancer immune escape. Thus, new strategies to overcome the immune suppressive tumor microenvironment (TME) are needed, and hence metabolic manipulation of T cells to optimally compete with cancer cells is an attractive strategy to restore anti-tumor immunity.

In one aspect disclosed herein are methods of treating, inhibiting, reducing, decreasing, amelioration, and/or preventing a cancer and/or metastasis (such as, for example, hepatocellular carcinoma and hematologic malignancies) in a subject, comprising administering to the subject a therapeutically effective amount of the therapeutic cells (such as, for example, any TIL, MIL, CAR T cell, CAR NK Cell, CAR NK T cell, CAR macrophage, and/or CAR B cell disclosed herein with reduced, inhibited, decreased, and/or ablated Sirt6 expression including, but not limited to any of the aforementioned cells further comprising reduced, inhibited, decreased, and/or ablated Sirt2 expression) disclosed herein.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

The disclosed treatments methods can also include the administration any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

Treatment methods can include or further include checkpoint inhibitors including, but not limited to, antibodies that block PD-1 (Pembrolizumab, Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

In some embodiments, a therapeutic agent for use in combination with chimeric cells for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF,

CD40L, Flt3 ligand, stem cell factor, ancestim, and TNFα. Suitable chemokines may include Glu-Leu-Arg (ELR)-negative chemokines such as IP-10, MCP-3, MIG, and SDF-1a from the human CXC and C-C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed chimeric cells are administered in combination with surgery.

As a consequence of the disclosed methods of inhibiting Sirt6 expression proliferation of immune cells and expression of effector molecules can be modulated. Accordingly, in one aspect, disclosed herein are methods of increasing proliferation, survival, and/or effector expression of an immune cell ((such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA). Also disclosed herein are methods of increasing effector molecule expression (such as, for example IFNγ and/or granzyme B) in an immune cell (such as, for example a tumor infiltrating lymphocytes (TILs), marrow infiltrating lymphocytes (MILs), T cell (including, but not limited to a CD4 T cell (including, but not limited to TH1 cells, TH2 cells, TH3 cells, TH17 cells, TH9 cells, TH22 cells) or CD8 T cell (including, but not limited to effector CD8 T cell, effector memory CD8 T cells, or central memory CD8 T cells) and γδ T cells), natural killer (NK) cell, NK T cell, macrophage, B cell, CAR T cell, CAR NK Cell, CAR NK T cell, CAR macrophage, and/or CAR B cells) comprising contacting the immune cell with a Sirt6 inhibitor or genetically modifying the lymphocytes to inhibit or ablate Sirt6 expression (such as, for example, a small molecule such as OSS_128167 (OSS), siRNA, shRNA, antisense oligonucleotide, or gRNA).

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Previous studies on Sirt6 epigenetic mechanisms have largely focused on glucose metabolism, tumor suppression and DNA repair in cancer cells. The work herein focuses on the epigenetic regulation of T cell metabolism by Sirt6, which has not been previously investigated. The work herein investigates chromatin remodeling via histone acetylation as a key epigenetic mechanism of immune regulation, governing T cell metabolic reprogramming via controlling the magnitude of the transcriptional activity of c-Myc and HIF-1α, the major regulators of T cell metabolism. Increasing the transcriptional activities of c-Myc and HIF-1α to improve immunotherapies has been elusive due to their pleiotropic functions and unintended toxicities. Blocking Sirt6's chromatin-silencer activity can unleash the transcriptional activity of c-Myc and HIF-1α specifically for the metabolic genes, thus offering a unique opportunity to manipulate T cell metabolism and augment cancer immunotherapy.

The work herein using metabolomic, molecular biology, epigenetic and immunologic methodologies mainly employ sorted primary T cells from mice and human samples, and thus delineates the endogenous expression and functions of Sirt6. Additional innovative methodology is provided by using CRISPR-Cas9 for genetic engineering of human primary TILs, immediately applicable for clinical translation.

### Mice

C57BL/6J mice, Pmel mice with a gp100-reactive transgenic T cell receptor (TCR): B6.Cg-Thyl^{a}/Cy Tg (TcraTcrb) 8Rest/J, major histocompatibility complex class II-restricted OVA-specific TCR (OT-II) transgenic mice: B6.Cg-Tg (TcraTcrb) 425Cbn/J were purchased from The Jackson Laboratory. All mice were bred and maintained under specific pathogen-free conditions at the animal facility of H. Lee Moffitt Cancer Center. All animal protocols were approved by the Institutional Animal Care and Use Committee (IACUC). Mice were used at 7-8 weeks of age.

### Mouse T Cell Culture

Spleens and/or lymph nodes (LN) were collected from C57BL/6J, Pmel and OT-II mice and were processed into single-cell suspensions. CD3+, CD4+ and CD8+ T cells were negatively enriched using Mouse Pan T cell, CD4⁺ T cell and CD8⁺ T Cell Isolation Kits™, respectively according to the manufacturer's instructions. The purity of the isolated cells was confirmed by flow cytometry (>95%). Purified T cells were cultured in complete RPMI 1640 medium (Thermo Fisher Scientific) supplemented with 10% (vol/vol) fetal bovine serum (FBS, Biowest) and 1% (vol/vol) penicillin/streptomycin (P/S, Thomas Scientific Inc.). T cells were activated with plate-bound anti-CD3 antibody (5 μg/ml, 145-2C11, BioXCell) for the indicated time periods. Pmel and OT-II splenocytes T cells were stimulated with gp100 (1 μg/ml, AnaSpec) and OVA-peptide (10 μg/ml, InvivoGen), and for the indicated time periods.

### Memory Differentiation

Pmel and OT-II splenocytes were activated with 1 μg/ml gp100 (AnaSpec) and 10 μg/ml ovalbumin 323-339 peptide (OVA-peptide, InvivoGen), respectively, for 3 days and subsequently cultured in the presence of 10 ng/ml IL-15 (R&D) for 4 days to generate T_{M} cells.

### Human PBMC

Human peripheral blood mononuclear cells (PBMC) were obtained from healthy donors by density gradient centrifugation using Ficoll-Paque™ PLUS Media (GE Healthcare). CD3+ lymphocytes were negatively enriched using human Pan T Cell Isolation Kit™ (Miltenyi Biotec). Enriched CD3+ T cells were cultured in complete RPMI medium and activated with plate-bound anti-CD3 antibody (5 μg/ml, OKT-3, BioXCell) in the presence of DMSO (vehicle), or the Sirt6 inhibitors, OSS (Selleck Chemicals), at the indicated concentrations.

### Human TILs

Human tumor infiltrating lymphocytes (hTILs) samples isolated from tumor biopsies of patients with non-small cell lung cancer (NSCLC) were generously provided by Moffitt Cancer Center. All human samples remained totally de-identified and no Institutional Review Board (IRB) approval was needed.

hTILs were maintained in RPMI 1640 Medium, supplemented with 10% human AB serum (Valley Biomedical, Inc.), 1 mM Hepes (Sigma-Aldrich), 1% P/S (Thomas Scientific Inc), 50 μg/ml Gentamicin (ThermoFisher Scientific), 50μM β-mercaptoethanol (ThermoFisher Scientific) and 6000 U/ml of recombinant human IL-2 (Peprotech). For metabolic and functional assays, hTILs were cultured in completed RPMI medium, IL-2 free, on plate-bound anti-CD3 antibody (5 μg/ml; OKT-3, BioXCell) in the presence of vehicle or OSS (Selleck Chemicals) at the indicated concentrations.

### Tumor Cells:

B16F10 melanoma cells, were obtained from the American Type Culture Collection. Cells were passaged minimally and maintained in complete Dulbecco's Modified Eagle Medium DMEM/F12 (Thermo Fisher Scientific) containing 10% FBS (Biowest) and 1% P/S (Thomas Scientific Inc).

### Method Details

### Subcutaneous Melanoma Tumor Model

Anesthetized mice were injected subcutaneously (s.c.) into the hind limb with 2.5×10⁵ B16F10 tumor cells in 200 μl of sterile PBS. Tumors were collected after 2 weeks and TILs were isolated for further analysis.

### Isolation of Mouse TILs

Lymphocytes from subcutaneous tumors were isolated by dicing the tissues followed by enzymatic digestion in PBS containing collagenase type 4 (2 mg/ml, Worthington Biochemical) and DNase I (0.25 mg/ml, Sigma-Aldrich) for 45 min with occasional shaking at 37° C. Cell suspensions were then twice filtered through 100 μm and 40 μm cell strainers (Thermo Fisher Scientific) to obtain single-cell suspension followed by a PBS wash and red blood cell lysis using RBC Lysis Buffer (Biolegend). TILs were finally isolated by density gradient centrifugation using Percoll (GE Healthcare) and were further purified using CD38 MicroBeads Kit™ (Miltenyi Biotec) according to the manufacturer's instructions. T cell purity was greater than 90% (data not shown). Fresh TILs were used directly for phenotypic and metabolic analyses or were cultured in complete RPMI medium for functional assays.

### Lentivector Transduction

Enriched CD3⁺ T cells from were stimulated for 24 hours in plate-bound anti-CD3 antibody (5 μg/ml, 145-2C11, BioXCell). Freshly concentrated lentivectors were spun-inoculated into activated T cells supplemented with polybrene (6 mg/ml, Sigma) at 2000 rpm, 32° C. for 2 hours.

### Flow Cytometry

For analysis of surface markers, cells were stained in PBS containing 5% FBS (vol/vol, FACS buffer) with: CD3 (BUV395), CD4 (BUV805), from BD Biosciences, CD8 (Alexa Fluor 700), CD44 (Alexa Fluor 488) and CD62L (PE-Cy7) from Biolegend, incubated at 4° C. for 1 hour, then washed twice with FACS buffer, and finally fixed in PBS containing 1% paraformaldehyde. Dead cells were excluded using the Zombie Violet™ Fixable Viability Kit (Biolegend) following the manufacturer's protocol.

For Sirt6 intracellular staining, cells were first labeled with surface markers CD4 (BUV 805), CD8 (Alexa Fluor 700), CD44 (Alexa Fluor 488), CD62L (PE-Cy7), and then fixed and permeabilized with Cytofix/Cytoperm™ Buffer (BD Biosciences). Intracellular labeling of Sirt6 was then performed using a specific Sirt6 antibody combined with a secondary Goat anti-rabbit IgG antibody (Alexa647, ThermoFisher) according to manufacturer's instruction.

For intracellular cytokine staining, cells were firstly stimulated with phorbol 12-myristate 13-acetate (PMA, 10 ng/ml, Sigma) and ionomycin (1 μM/ml, Sigma) for 1 hour followed by GolgiPlug™ treatment (1% c, BD Biosciences) for 6 h and then stained with IFN-γ (BV711, BD Biosciences) and Granzyme B (Alexa647, Biolegend). Cells were acquired on a BD FACSymphony™ A5 and LSR II (Becton Dickinson), and data were analyzed with FlowJo software Version 10.0 software.

### CFSE Proliferation Assay

Pmel cells and OT-II cells were first labelled with 5 μM of CellTrace™ carboxyl fluorescein succinimidyl ester (CFSE, Thermo Fisher Scientific) for 30 min at room temperature (RT) and then stimulated with gp100 or remained non-stimulated with and without Sirt6 inhibitor (OSS) for 3 days.

### Enzyme-Linked Immunospot (ELISPOT) Assay

The number of IFN-γ producing cells was evaluated in an ELISPOT assay. Briefly, mouse TILs were plated at 1×10⁵ cells and co-cultured with 1×10³ irradiated B16F10 tumor cells (50 Gray) in the presence of the indicated concentrations of OSS, in a 96-well nitrocellulose flat-bottomed plate (ELISPOT plate, Millipore) pre-coated with an anti-mouse IFN-γ antibody (AN-18, Mabtech, Inc.) and incubated for 48 hr at 37° C. For Pmel T cells, splenocytes were first stimulated with gp100 (1 μg/ml, Anaspec) for 48 hr in the presence of the indicated concentrations of OSS and then transferred to the ELISPOT plate pre-coated with an anti-mouse IFN-γ antibody (AN-18, Mabtech, Inc.) for an additional 48 hr. For human CD3+ T cells and TILs, cells were first stimulated with plate-coated anti-CD3 for 48 hr in the presence of vehicle or OSS at the indicated concentration and then transferred to the ELISPOT plate pre-coated with an anti-human IFN-γ antibody (1-DIK, Mabtech, Inc.). PMA (10 ng/ml, Sigma-Aldrich) was used as positive assay control. After washing steps and successive biotinylated anti-IFN-γ antibody (R4-6A2, Mabtech, Inc.) and Streptavidin-HRP (BD Bioscience) labeling, spots were detected using AEC Substrate Kit (BD Biosciences) and counted using an AID ELISPOT Reader System (Autoimmun Diagnostika GmbH).

### Lactate Dehydrogenase (LDH) Release Assay

Evaluation of functional cytotoxic activity was performed using Pierce™ LDH Cytotoxicity Assay Kit (Thermo Fisher Scientific) according to the manufacturer's protocol. B16F10 cells or human NSCLC autologous tumor cells were used as target cells. Mouse CD8+ enriched T cells from gp100-stimulated Pmel splenocytes or human NSCLC TILs were used as effector cells. Target tumor cells were co-cultured with effector cells in 96-well round-bottom microplates at the indicated effector:target ratios with and without Sirt6 inhibitor. After 6 hr, the percentage of specific LDH release was calculated according to the following formula: % cell lysis=(experimental value−effector cells spontaneous release−target cells spontaneous release)×100/(target cells maximum release−target cells spontaneous release), where “experimental” corresponds to the experimental signal value, “effector spontaneous” to spontaneous release of LDH from effector cells alone, “target spontaneous” to spontaneous release of LDH from target cells alone and “maximum release” to the maximum release of LDH from target cells in medium containing 1% Triton X-100.

### Extracellular Flux Analysis

Extracellular acidification rate (ECAR) and oxygen consumption rate (OCR) were measured using the Seahorse XF analyzer (Agilent technologies). Glycolysis Stress Test (GST) and mitochondrial stress test (MST) were purchased from Agilent technologies and performed according to the kit manufacturer's instructions. The day of the assay, 2×10⁵ activated T cells were plated in Cell-Tak (Corning)-coated XF96 plates in XF media (XF RPMI Medium pH 7.4 containing 2 mM L-glutamine only for GST or supplemented with 10 mM glucose, 2 mM L-glutamine and 1 mM sodium pyruvate for GRA and MST, all from Agilent technologies) via centrifugation to ensure cell attachment. Cells were equilibrated for 1 hr in a non-CO₂ incubator before starting the assay. For GST, ECAR and OCR were measured under basal conditions and in response to 10 mM glucose, 1 μM oligomycin and 50 mM 2-DG successively to calculate basal glycolytic rate, glycolytic capacity (in response to oligomycin), and glycolytic reserve (glycolytic capacity-basal rate). For MST, OCR and ECAR were measured under basal conditions and in response to sequential injections of 1 μM oligomycin, 1 μM FCCP (Carbonyl cyanide 4-trifluoromethoxyphenylhydrazone) and 0.5 μM rotenone/antimycin A to calculate basal respiration rates (baseline OCR-rotenone/antimycin A OCR), maximal respiration (FCCP OCR-rotenone/antimycin A OCR), and oxidative reserve (maximum respiration rate-basal respiration rate). Results were normalized to total protein quantified using Pierce™ BCA Protein Assay Kit (Thermo Fisher Scientific).

### Immunoprecipitation

For immunoprecipitation (IP) assays, activated CD3⁺ T cells were lysed in IP lysis buffer [20 mM Hepes, pH 7.9, 180 mM KCl, 0.2 mM EDTA, 1.5 mM MgCl₂, 20% (vol/vol) glycerol, 0.1% Nonidet P-40, containing a mixture of protease inhibitors]. Cell lysates were incubated at 4° C. overnight with specific antibodies against Sirt6, c-Myc HIF-1α and H3K9 (Cell signaling). Rabbit mAb IgG (Cell Signaling, #3900) was used as an isotype control mAb. After addition of anti-rabbit Ig agarose-beads (TRUEBLOT®, Rockland), samples were incubated at 4° C. for 2 hours. Beads were washed five times with IP lysis buffer and proteins were released from the beads by boiling in 3× SDS sample loading buffer and loaded into 10% SDS-PAGE gel for Western blot analysis.

### Western Blot Analysis

Whole cell lysates were prepared using lysis buffer (Pierce RIPA buffer, Thermo Fisher Scientific) supplemented with Complete™ protease inhibitor cocktail and PhosSTOP phosphatase inhibitor cocktail (Roche Applied Science).

Cell lysates (20 μg) or IP samples were loaded onto 10% SDS-PAGE and separated by electrophoresis followed by semi-dry transfer into polyvinylidene fluoride membranes (Immun-Blot® PVDF membrane, Bio-Rad) using Trans-Blot Turbo transfer system (Bio-Rad). After transfer, the membranes were blocked at room temperature with Tris-buffered saline (TBS) containing 0.05% Tween-20 (TBST) and 5% (w/v) non-fat dry milk for 1 hour and then incubated overnight at 4° C. with a specific primary antibody (indicated below). The membranes were washed three times with TBST and then incubated for 1 hour with horseradish peroxidase (HRP)-conjugated goat anti-mouse IgG H&L (Abcam, #ab97051, 1:3,000) for regular western blotting analysis, or HRP-conjugated mouse anti-rabbit IgG light-chain specific (Cell Signaling, #93702, 1:1,000) for IP samples. After washing 3 times with TBST, bound antibodies were detected by chemiluminescence using the Pierce™ ECL Western Blotting Substrate and the Super Signal West Femto Maximum Sensitivity Substrate kits (Thermo Fisher Scientific). Image acquisition was performed with the Amersham imager 600 system (GE Healthcare Bio-Sciences).

Immunoblotting was performed using primary antibodies against: Sirt6, H3K9, acetylated-H3K9 and c-Myc (#9402, 1:1,000) from Cell signaling, LDH (#ab52488, 1:5,000), PFKP (#ab204131, 1:2,000), HK1 (#ab150423, 1:1,000), HIF-1α and beta-Actin from Abcam

### RT-PCR

Total RNA was extracted using the RNeasy Mini Kit (Qiagen; cat. #74106) and reverse transcribed using iScript™ (Biorad). cDNAs were amplified using the “SsoAdvanced™ Universal SYBR® Green Supermix” kit (Biorad) in the CFX Connect™ Real-Time System (Biorad). Sense and antisense primers used were for mouse Sirt6, GLUT1, HK, PFK, LDH, GLS and ASCT2 (primers designed by Biorad and provided as a mixture, PrimePCR™ SYBR® Green Assay).

### Sirt6 Deletion by CRISPR-Cas9

Primary human CD3+ T cells from healthy donors were activated for 48 hours and then electroporated with vehicle (Ctl), Cas9-Atto550-crRNA non targeting control (NTC) or Cas9-Atto550-crRNA targeting Sirt6 (crSirt6). 72 hours after electroporation Atto550+ T cells were sorted and then expanded for 9 days. Detection of Sirt6 mutant was performed by PCR and western blot.

### 1. Example 1: Sirt6 Expression is Induced in T Cells Upon Activation, Maturation and within the TME

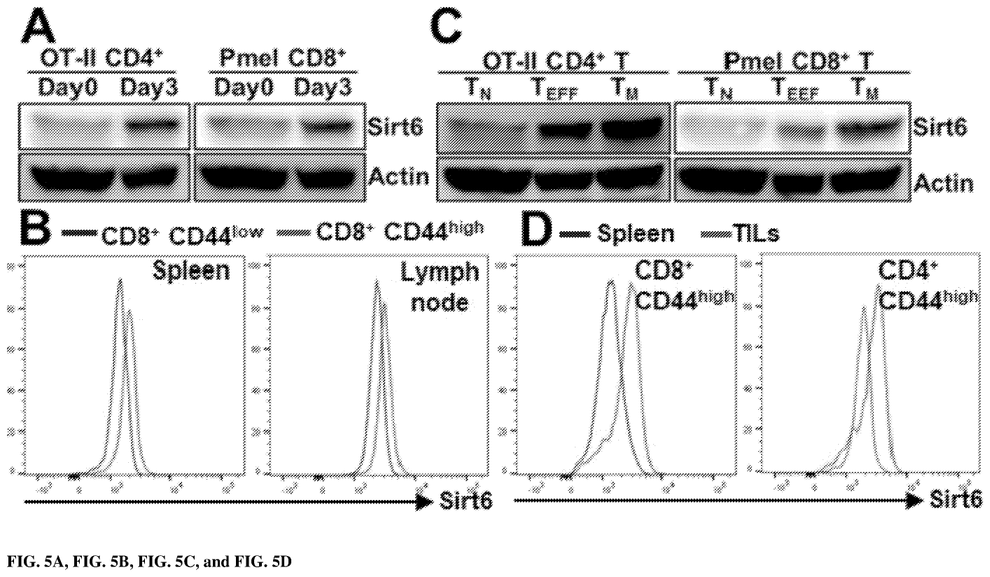

To gain insight into the role of Sirt6 in T cell homeostasis, Sirt6 expression was analyzed following T cell activation and maturation. Interestingly, we found that Sirt6 expression was induced ex vivo upon T cell receptor (TCR) stimulation with cognate antigenic peptides in OT-II (OVA323-339) and Pmel (gp10025-33) T cells (FIG. 5A). Additionally, Sirt6 was differentially expressed among T cell subsets, prominently upregulated in TM cells (CD44high) in spleen and lymph node (FIG. 5B). Upregulation of Sirt6 expression in TM cells was confirmed ex vivo by WB on IL-15 differentiated OT-II and Pmel™ cells (FIG. 5C). Strikingly, using preclinical melanoma mouse models, we found Sirt6 upregulated in CD8+ and CD4+ TILs isolated from subcutaneous (s.c.) B16F10 nodules when compared to the peripheral splenic counterpart cell populations (FIG. 5D). This result may be related to the fact that Sirt6 expression is highly regulated by nutrient availability. Therefore, Sirt6 might be induced in TILs within the TME where glucose is scarce.

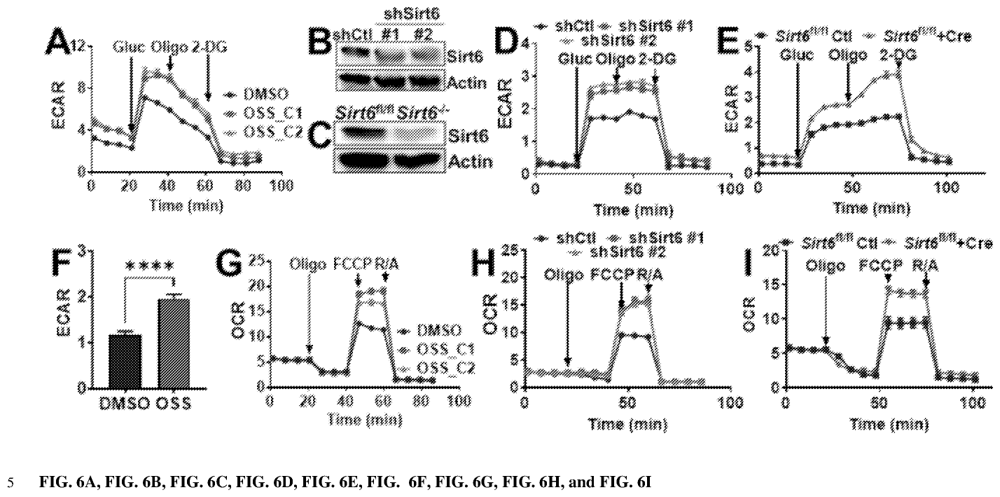

Given that Sirt6 expression is induced upon T cell activation, and Sirt6 is a master regulator of glucose metabolism in other cell types, we then investigated whether Sirt6 inhibition in T cells alters their glycolytic and mitochondrial activities during activation by measuring extracellular acidification rate (ECAR) and oxygen consumption rate (OCR) using the Seahorse extracellular flux analyzer. Interestingly, Sirt6 inhibition using OSS_128167 (OSS), a specific Sirt6 inhibitor, and Sirt6 gene knockdown using shRNA lentivectors as well as Sirt6 gene knockout using Sirt6f1/fl mice profoundly increased the glycolytic flux of activated CD3+ T cells (FIG. 6A-E) when compared to the controls. Consistent with this finding, Sirt6 inhibition restored the glycolytic activity of TILs isolated from s.c. B16F10 melanoma tumors in C57BL/6 mice (FIG. 6F). Furthermore, our data indicate increase in the SRC in response to the mitochondrial decoupling agent (FCCP) similarly with Sirt6 pharmacologic inhibition, gene knockdown and knockout in activated T cells (FIG. 6G-I).

### 2. Example 2: Sirt6 Inhibition Improves T Cell Effector Functions

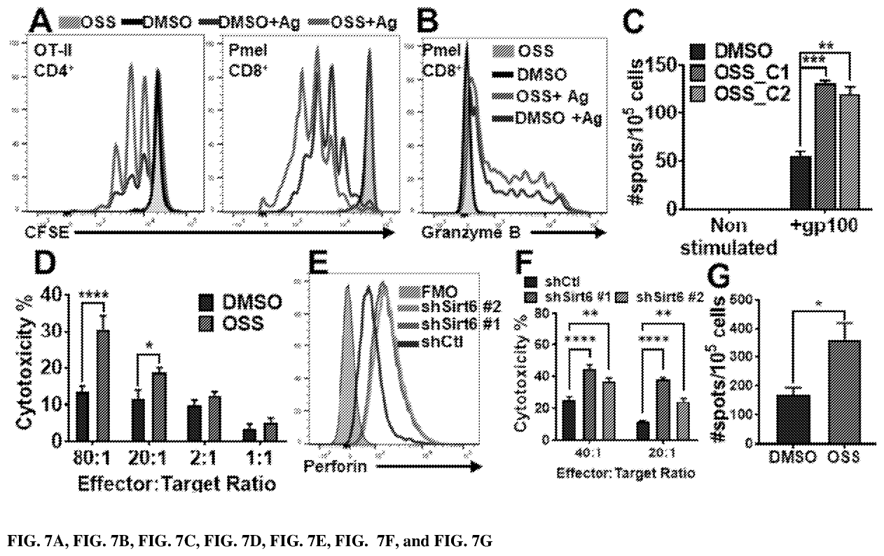

Given that aerobic glycolysis is specifically required for effector function in T cells, we then investigated whether enhanced glycolysis with Sirt6 inhibition influences T cell functions. These studies revealed that Sirt6 inhibition increased proliferation of OT-II and Pmel T cells upon antigenic stimulation ex vivo (FIG. 7A). Moreover, activated Pmel T cells demonstrated increases in granzyme B expression and IFN-γ release with the Sirt6 inhibitor (FIG. 7B&C). This hyper-reactive phenotype of Pmel T cells with Sirt6 inhibition led to superior cytotoxic activity against B16F10 cell challenge ex vivo (FIG. 7D). Increases in effector molecule expression (FIG. 7E) and killing capability (FIG. 7F) of Pmel T cells were also manifest using Sirt6 gene knockdown. More importantly, when TILs isolated from B16F10 tumors were treated with the Sirt6 inhibitor and re-challenged with B16F10 cells ex vivo, they exhibited an increase in IFN-γ release (FIG. 7G). These data suggest that Sirt6 upregulation limits the effector activity and the proliferative capacity of antigen-stimulated T cells.

### 3. Example 3: Sirt6 Interacts with c-Myc and HIF-La and Regulates the Transcription of Key Glycolytic Targets

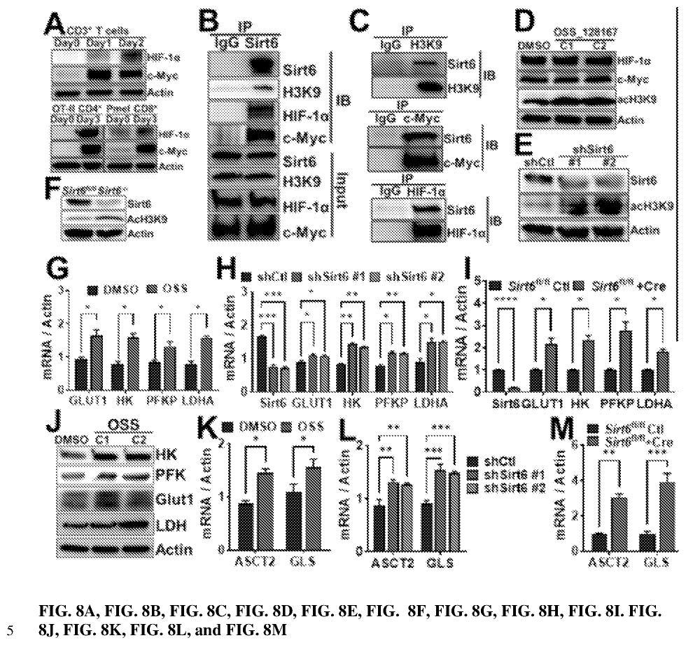

c-Myc and HIF-1α expression is induced following TCR ligation (FIG. 8A) concomitantly with Sirt6 induction (FIG. 5A) Prior studies indicate that Sirt6 binds to c-Myc and HIF-1α and co-represses their transcriptional activity by deacetylating histone H3K9 at the promoter of their respective target genes. Therefore, we investigated whether similar mechanisms are manifest in T cells. Strikingly, Sirt6 interactions with c-Myc, HIF-1α and histone H3K9 were confirmed in activated T cells by immunoprecipitation-immunoblot (IP-IB) and reverse IP-IB assays (FIG. 8B&C). Importantly, Sirt6 inhibition with either drug or gene silencing or knockout resulted in increased H3K9 acetylation levels (FIG. 8D-F), with no effect on c-Myc and HIF-1α expression levels (FIG. 8D), confirming the chromatin-silencer activity of Sirt6 in T cells.

c-Myc and HIF-1α coordinately activate the transcription of genes required for aerobic glycolysis and glutaminolysis in activated T cells. Therefore, we analyzed the impact of Sirt6 blockade on the expression levels of the glycolytic and glutaminic enzymes. Our results indicate that Sirt6 inhibition with either drug or gene knockdown or knockout in activated T cells increased mRNA expression levels of glucose transporter (Glut1), and key glycolytic enzymes including hexokinase (HK1), phosphofructokinase P (PFKP) and lactate dehydrogenase A (LDHA) (FIG. 8G-I). Consistently, WB analysis confirmed the upregulation of Glut1, HK1, PFKP and LDHA protein expression levels with Sirt6 inhibition (FIG. 8J). Furthermore, Sirt2 targeting also increased the mRNA expression levels of glutamine transporter (ASCT2) and glutaminase (GLS) (FIG. 8K-M). These observations provide mechanistic rational for increased glycolytic flux and SRC resulted with Sirt6 targeting, and strongly suggest Sirt2 as an epigenetic regulator of T cell metabolism.

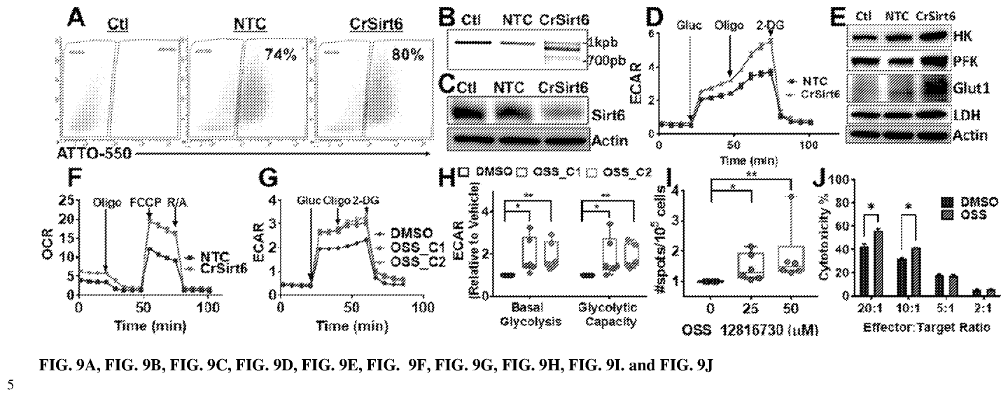

To determine the translational potential of Sirt6 blockade, primary human T cells from healthy donors were gene-edited using CRISPR/Cas9 technology to delete Sirt6 (FIG. 9A-C) and then the impact of Sirt6 deletion was examined. Consistently with our mouse studies, Sirt6 deletion in human CD3+ T cells increased the glycolytic rate, and the expression levels of key glycolytic targets as well as the mitochondrial SRC (FIG. 9D-F). Further, human TILs isolated from 6 NSCLC patient tumor samples treated with Sirt6 inhibitor also exhibited increased aerobic glycolysis and IFN-γ production (FIG. 9G-I). More importantly, when patient-derived human NSCLC tumor cells were co-cultured with ex vivo expanded autologous TILs, the Sirt6 inhibitor enhanced TIL cytotoxic activity (FIG. 9J). Therefore, Sirt6 targeting emerges as a promising intervention to enhance the metabolic fitness and effector functions of tumor-reactive T cells.

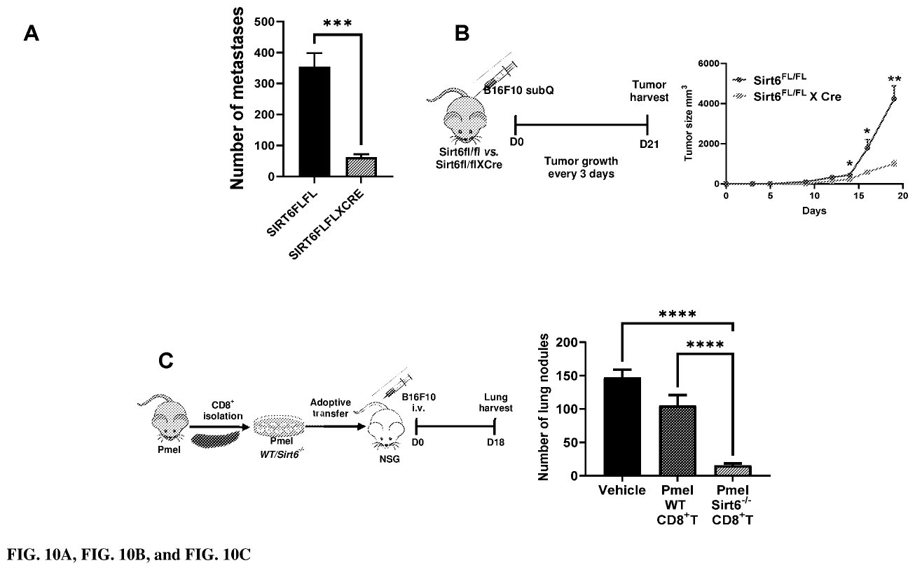

To determine the effect of Sirt6 blockade on anti-tumor immunity, Sirt6fl/fl and Sirt6f1/f1XCre mice were challenged with B16F10 cells and lung metastatic nodules measured (FIG. 10). Results showed that the number of metastases was approximately 7-fold less in the Sirt6f1/f1XCre mice. Similarly, tumor size was considerably smaller (at least 5-fold) in the Sirt6fl/f1XCre mice compared to Sirt6fl/fl (FIG. 10B). Next, CD8+ T cells from wildtype (WT) Pmel mice or Sirt6−/− Pmel mice were transferred to NSG mice. The recipient NSG mice were then challenged with B16F10 cells and the number of lung metastatic nodules measured (FIG. 10C). It was observed that mice receiving T cells from the Pmel Sirt6−/− mice had 5-10-fold fewer lung nodules than mice receive T cells from Pmel WT mice or vehicle alone.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. The method making a modified immune cell comprising
(a) collecting lymphocytes from a subject with cancer; and
(b) treating the lymphocytes with a Sirt6 inhibitor or genetically modifying the lymphocytes to inhibit or ablate Sirt6 expression.

2. The method of claim 1, wherein the Sirt6 inhibitor is OSS_128167 (OSS), an siRNA, shRNA, antisense oligonucleotide, or gRNA.

3. (canceled)

4. The method of claim 1, wherein the lymphocytes are genetically modified by inserting a chimeric receptor into the genome of the cell at a location that disrupts expression or activity of an endogenous Sirt6 protein.

5. The method cell of claim 4, wherein the chimeric receptor is a chimeric antigen receptor (CAR) polypeptide.

6. The method of claim 1, wherein the modified immune cell is further modified by treating the lymphocytes with a Sirt2 inhibitor or genetically modifying the lymphocytes to inhibit or ablate Sirt2 expression

7. The method of claim 6, wherein the Sirt2 inhibitor is AGK2, AK-1, SirReal2, Tenovin-6, Thiomyristoyl, AEM1, AEM2, an siRNA, antisense oligonucleotide, or gRNA.

8. (canceled)

9. The method of claim 6, wherein the lymphocytes are genetically modified by inserting a chimeric antigen receptor into the genome of the cell at a location that disrupts expression or activity of an endogenous Sirt2 protein.

10. (canceled)

11. The method of claim 1, wherein the lymphocytes are tumor infiltrating lymphocytes (TILs).

12. A therapeutic cell produced by the method of claim 1.

13. A method of treating a cancer in a subject, comprising administering to the subject a therapeutically effective amount of the therapeutic cells of claim 12.

14. The method of claim 13, further comprising administering to the subject an anticancer agent.

15. The method of claim 14, wherein the anticancer agent is a checkpoint inhibitor.

16. The method of claim 15, wherein the checkpoint inhibitor comprises an anti-PD-1 antibody, anti-PD-L1 antibody, anti-CTLA-4 antibody, or any combination thereof.

17. A method of increasing proliferation and/or survival of an immune cell; a method of increasing glycolysis, mitochondrial respiration, glutaminolysis, fatty acid oxidation, lipogenesis metabolism of an immune cell; a method of increasing the transcription of glycolytic targets, glutaminolysis pathways targets and fatty acid oxidation targets in an immune cell; or a method of increasing the transcriptional activity of c-Myc and HIF-1α transcription factors in an immune cell; the method comprising contacting the immune cell with a Sirt6 inhibitor or genetically modifying the immune cell to inhibit or ablate Sirt6 expression.

18. (canceled)

19. (canceled)

20. (canceled)

21. The method of claim 17, wherein the immune cell comprises a CD8+ or CD4+ T cell.

22. The method of claim 17, wherein the Sirt6 inhibitor is OSS_128167 (OSS), an siRNA, shRNA, antisense oligonucleotide, or gRNA.

23. (canceled)

24. A method of increasing effector molecule expression in a T cell an immune cell comprising contacting the immune cell with a Sirt6 inhibitor or genetically modifying the immune cell to inhibit or ablate Sirt6 expression.

25. The method of claim 24, wherein the effector molecule comprises granzyme B or IFNγ.

26. The method of claim 24, wherein the immune cell comprises a CD8+ or CD4+ T cell.

27. The method of claim 24, wherein the Sirt6 inhibitor is OSS_128167 (OSS), an siRNA, shRNA, antisense oligonucleotide, or gRNA.

28. (canceled)
